(19) United States
(12) Patent Application Publication
(10) Pub. No.: US 2020/0113836 A1
(43) Pub. Date: Apr. 16, 2020

(54) METHOD OF PREPARING SUSTAINED-RELEASE DRUG MICROPARTICLES WITH EASY RELEASE CONTROL

(71) Applicant: DAEWOONG PHARMACEUTICAL CO., LTD., Hwaseong-si, Gyeonggi-do (KR)

(72) Inventors: Byoung Chan BAE, Yongin-si, Gyeonggi-do (KR); Sung Hoon PARK, Yongin-si, Gyeonggi-do (KR); Tae Ho LEE, Yongin-si, Gyeonggi-do (KR)

(21) Appl. No.: 16/618,219

(22) PCT Filed: May 21, 2018
(86) PCT No.: PCT/KR2018/005785

§ 371 Date: Nov. 29, 2019

(30) Foreign Application Priority Data
May 31, 2017 (KR) 10-2017-0067639

(51) Int. Cl. A61K 9/16; A61K 9/107; A61K 31/445; A61K 47/34

(52) U.S. Cl. CPC A61K 9/1682; A61K 47/34; A61K 31/445; A61K 9/107

(57) ABSTRACT

The present invention relates to a method of preparing sustained-release drug microparticles allowing easy release control. According to the method of the present invention, as can be seen in Examples of the present invention, an initial release amount may be easily controlled by simply adjusting the evaporation temperature of a solvent in a conventional method of preparing microparticles. In addition, since no additional process is required, a drug loading rate may be significantly increased, and since high temperature is not required, the stability of a drug that is weak to heat may be ensured.

### TECHNICAL FIELD

The present invention relates to a method of preparing sustained-release drug microparticles allowing easy release control.

### BACKGROUND ART

For sustained release of drugs, techniques for loading drugs into biodegradable polymer microparticles have been developed. However, when these techniques are applied to drugs prepared in the form of particulates, there is a problem that initial release of the drugs is excessive.

In general, microparticles are prepared using a solvent evaporation method, a spray drying method, a coacervation method, or the like. Thereamong, the solvent evaporation method is the most widely used.

In the solvent evaporation method, for example, an O/W emulsion or a W/O/W emulsion is prepared, and then a solvent is evaporated from the emulsion to form microparticles. The most common method for solvent evaporation is to remove a solvent by raising temperature to near the boiling point of the solvent. However, in the case that the boiling point of a volatile solvent is close to the glass transition temperature (Tg) of a polymer, when the volatile solvent begins to volatilize as temperature increases to the boiling point of the volatile solvent, deformation may occur in the crystal form of the polymer, and pores may be formed on the surfaces of microparticles, which may change a release rate. That is, in this case, an in vitro release rate may be increased, and side effects may occur in vivo.

Referring to the prior art, Patent Document 1 (Korean Patent No. 10-1481859) discloses a process of obtaining coagulated polymer microparticles from an emulsion and treating the microparticles with an aqueous alcohol solution. According to Patent Document 1, aqueous alcohol solution treatment reduces the Tg of a polymer to TgA, thereby reducing the internal pore structure of microparticles. As a result, the particles are densified, which reduces initial release of a drug. However, since the polymer microparticles may be lost in recovery and drying processes performed after the aqueous alcohol solution treatment, further processing is required. In addition, when a polymer having high Tg is used, it is necessary to apply a temperature close to or higher than the Tg. Thus, for heat-sensitive pharmaceutical active ingredients, stability may not be guaranteed.

Patent Document 2 (Korean Patent No. 10-1583351) discloses microparticles prepared by loading a drug into a carrier formed of a biodegradable polymer. According to Patent Document 2, by additionally introducing an initial recovery process to a solvent exchange evaporation method using a co-solvent, initial excessive release of a biologically active substance may be suppressed, and the removal rate of a residual solvent may be increased. In addition, in an initial recovery process, methylene chloride, a hydrophobic solvent, may not be easily released to the outside through the surfaces of microparticles that are not completely cured, but dimethylsulfoxide, an amphiphilic solvent, may be easily released to the outside. However, also in Patent Document 2, since the hydrophobic solvent should be removed through evaporation, a conventional method of removing a residual solvent by increasing temperature to the boiling point of the solvent is used. Accordingly, the above-mentioned problem is caused when volatilization occurs as temperature is increased to the boiling point of the volatile solvent.

Therefore, there is demand for a method of preparing sustained-release drug microparticles that exhibit pharmacodynamics that do not allow excessive increase in initial release of a drug and do not exceed the blood drug concentration of an oral drug while solving the problems of the prior art.

### DISCLOSURE

### Technical Problem

Therefore, the present invention has been made in view of the above problems, and it is one object of the present invention to provide a method of preparing sustained-release drug microparticles that exhibit pharmacodynamics that do not allow excessive increase in initial release of a drug and do not exceed the blood drug concentration of an oral drug.

### Technical Solution

In accordance with one aspect of the present invention, provided is a method of preparing sustained-release drug microparticles, the method comprising:

mixing a mixed solution of a drug and a biodegradable polymer dissolved in a solvent with an aqueous medium to obtain an emulsion; and

evaporating the solvent from the emulsion to form microparticles containing the drug,

wherein evaporation of the solvent is performed by heating at a rate of 0.2 to 2° C./min so that temperature increases from a temperature before the solvent evaporation to a temperature in a range of a boiling point ±10° C. of the solvent.

The present invention is similar to conventional methods in that an emulsion is prepared and microparticles are obtained from the emulsion, but the present invention is characterized in that solvent evaporation is performed by slowly heating so that temperature increases from a temperature before a solvent evaporation step to a temperature in a range of a boiling point ±10° C. of a solvent.

According to the method of preparing sustained-release drug microparticles according to the present invention, the initial drug release rate of the sustained-release drug microparticles may be significantly reduced. The initial drug release rate may be determined relatively, depending on the total drug release period of the sustained-release microparticles. In the present invention, an initial drug release rate refers to a drug release rate during a period corresponding to, for example, the initial ⅙ to ⅓ of a total release period from administration of the sustained-release microparticles to complete release of the drug, without being limited thereto. As can be seen in Examples below, for example, for a one-month release formulation, an initial drug release rate may refer to the proportion of drug released from sustained-release microparticles within about 7 days. In this case, the initial drug release rate may be determined using the concentration of a drug initially loaded in the sustained-release microparticles and the amount of the drug remaining in the sustained-release microparticles as measured at a specific time point (e.g., 7 days after administration) within 7 days after administration.

Examples below show that microparticles obtained through solvent evaporation performed at various temperatures exhibit different initial drug release rates. Based on these results, the present inventors used a method of slowly heating so that temperature increases from a temperature before a solvent evaporation step to a temperature in a range of a boiling point ±10° C. of a solvent as a method of controlling an initial drug release rate. An in vivo PK test using microparticles prepared according to the method of the present invention confirmed that initial drug release was controlled in animals.

For example, the initial release rate of the sustained-release drug microparticles prepared according to the present invention may be less than 50%, less than 40%, or less than 30%, for one-month formulation, as measured at a specific time point within 7 days after administration (e.g., 7 days after administration), without being limited thereto.

Drug release of the sustained-release drug microparticles according to the present invention may last several weeks or months. The duration of release of a drug may be determined depending on the loading amount of a drug, the type of a biodegradable polymer, a blending ratio, the content of additives, and the like in preparing microparticles, and the related art is well known to those skilled in the art. Thus, those skilled in the art may appropriately design a drug release duration and a release rate according to the type of drug to be administered to a patient, dosage, an administration method, disease severity, and the like.

When a pharmaceutical formulation comprising the sustained-release drug microparticles according to the present invention is administered, the maximum blood concentration of a drug does not exceed the maximum drug concentration in blood at the time of administration of an oral pharmaceutical formulation in a dose corresponding to the formulation. For example, in the case of using a pharmaceutical formulation (drug dose of 300 mg) containing one-month drug release-type sustained-release drug microparticles in place of an oral pharmaceutical formulation used in a drug dose of 10 mg once a day, when the pharmaceutical formulation containing the sustained-release drug microparticles according to the present invention is administered, the maximum drug concentration in blood of the pharmaceutical formulation does not exceed the maximum drug concentration in blood at the time of administration of the oral pharmaceutical formulation corresponding thereto.

That is, in comparison with an oral pharmaceutical formulation wherein an active ingredient dose identical to that of the pharmaceutical formulation comprising the sustained-release drug microparticles according to the present invention is administered multiple times, the pharmaceutical formulation comprising the sustained-release drug microparticles may exhibit a maximum drug concentration in blood (Cmax) that is biologically equivalent to that of the oral pharmaceutical formulation. Here, whether maximum drug concentration in blood (Cmax) represents a bioequivalent level may be determined according to drug equivalence criteria. For example, in accordance with the bioequivalence test of drug equivalence test criteria specified in the Pharmaceutical Affairs Law, when the maximum drug concentrations in blood (Cmax) of each of a reference drug and a test drug are log-converted and analyzed statistically, it is assumed to be biologically equivalent when the analyzed value satisfies a range of log 0.8 to log 1.25 in a 90% confidence interval of log-converted mean difference.

In addition, the temperature before a solvent evaporation step may refer to room temperature or a temperature measured at the start of solvent evaporation after preparation of an emulsion.

The range of a boiling point ±10° C. of a solvent is given because a final target temperature upon heating depends on the type of a solvent used. Since volatilization of a solvent occurs near the boiling point of the solvent, the range of a boiling point ±10° C. of a solvent is used. The range may include a range of a boiling point ±10° C. of a solvent, a range of a boiling point ±8° C. of a solvent, a range of a boiling point ±6° C. of a solvent, and a range of a boiling point ±4° C. of a solvent, a range of a boiling point ±2° C. of a solvent, and the like, and include all sub-values within the ranges.

Heating may be performed at a heating rate of 0.2 to 2° C./min, for example, 0.3 to 1.5° C./min or 0.5 to 1° C./min, so that temperature increases from a temperature before a solvent evaporation step to a temperature in a range of a boiling point ±10° C. of a solvent.

According to the preparation method of the present invention, as can be seen in Examples below, an initial release amount may be easily controlled by simply adjusting the evaporation temperature of a solvent in a conventional method of preparing microparticles. In addition, since no additional process is required, a drug loading rate may be significantly increased, and since high temperature is not required, the stability of a drug that is weak to heat may be ensured.

In one embodiment of the present invention, N₂ may optionally be fed during solvent evaporation. Since N₂ promotes evaporation of a solvent, N₂ treatment may be performed in the solvent evaporation step as needed.

In one embodiment of the present invention, the biodegradable polymer may be selected from the group consisting of polylactide (PLA), polyglycolide (PGA), poly(lactide-co-glycolide) (PLGA), and mixtures thereof.

The ratio of polylactide to polyglycolide in the poly(lactide-co-glycolide) copolymer may be 50:50 to 95:5, for example, 50:50, 65:35, 75:25, or 85:15.

The biodegradable polymer may have a weight average molecular weight of 4,000 to 50,000, without being limited thereto. For example, the weight average molecular weight of the biodegradable polymer may be 4,000 to 15,000, 7,000 to 17,000, 5,000 to 20,000, 10,000 to 18,000, or 18,000 to 28,000, and include all sub-values within the ranges.

For example, as the biodegradable polymer used in the present invention, polylactide, polyglycolide, poly(lactide-co-glycolide) under RESOMER™ from Evonik Rohm GmbH Co. may be used, or these polymers are blended and used. For example, R202H, R202S, R203H, R203S, RG502H, RG503H, RG653H, RG752H, RG752S, RG753H, RG753S, or a blended polymer thereof may be used. In Examples below, RG203H, RG502H, RG752H, or a biodegradable polymer obtained by blending polylactide and these polymers is used.

A molecular weight of a biodegradable polymer or a blending ratio may be appropriately selected by those skilled in the art in consideration of the decomposition rate of the biodegradable polymer, a drug release rate, and the like.

The type of a drug loaded in the sustained-release drug microparticles of the present invention is not particularly limited, and may include poorly soluble drugs. The basic principle of loading a poorly soluble drug into the sustained-release drug microparticles is to use hydrophobic bonding. That is, hydrophobic interaction between the hydrophobic portion of a biodegradable polymer to be used and the hydrophobic portion of a poorly soluble drug is generated, resulting in the poorly soluble drug being loaded. Accordingly, common poorly soluble drugs may be wrapped by the hydrophobic portion of a polymer during emulsification. At this time, cohesion is inversely proportional to the solubility of a poorly soluble drug. As described above, since the molecular weight of the biodegradable polymer used in the present invention is 4,000 to 50,000 and the molecular weight of common poorly soluble drugs is less than 2,000, the drugs may be sufficiently loaded in the polymer. Accordingly, all of common poorly soluble drugs may be loaded in the sustained-release drug microparticles according to the present invention.

The drug loaded in the sustained-release drug microparticles may include one or more selected from the group consisting of progesterone, haloperidol, thiothixene, olanzapine, clozapine, bromperidol, pimozide, risperidone, ziprasidone, diazepam, ethyl loflazepate, alprazolam, nemonapride, fluoxetine, sertraline, venlafaxine, donepezil, tacrine, galantamine, rivastigmine, selegiline, ropinirole, pergolide, trihexyphenidyl, bromocriptine, benztropine, colchicine, nordazepam, etizolam, bromazepam, clotiazepam, mexazolam, buspirone, goserelin, leuprolide, octreotide, cetrorelix, fluconazole, itraconazole, mizoribine, cyclosporin, tacrolimus, naloxone, naltrexone, cladribine, chlorambucil, tretinoin, carmustine, anagrelide, doxorubicin, anastrozole, idarubicin, cisplatin, dactinomycin, docetaxel, paclitaxel, raltitrexed, epirubicin, letrozole, mefloquine, primaquine, oxybutynin, tolterodine, allylestrenol, lovastatin, simvastatin, pravastatin, atorvastatin, alendronate, raloxifene, oxandrolone, estradiol, ethinylestradiol, etonogestrel, and levonorgestrel, without being limited thereto.

In one embodiment of the present invention, the drug may be donepezil. Currently, Aricept™ Tab (EISAI Co.), a donepezil-containing oral tablet, is taken once a day before bedtime, and 5 mg, 10 mg, 23 mg tablets are commercially available. However, oral administration of donepezil-containing oral tablets is known to cause gastrointestinal side effects such as diarrhea, nausea, loss of appetite, and muscle convulsion in some patients. In addition, since patients with Alzheimer's disease have to take the drug repeatedly before bedtime once a day, the convenience of taking the drug may be reduced, and thus it may be difficult to obtain a continuous pharmacological effect. Therefore, when an injection is prepared by loading donepezil in the sustained-release drug microparticles of the present invention and the injection is used, patient convenience of taking a drug may be improved, and at the same time, a continuous pharmacological effect may be obtained.

In the method of preparing sustained-release drug microparticles according to the present invention, the solvent may be a volatile solvent. The solvent is used to dissolve a polymer or a drug. However, the solvent remaining in the microparticles may cause problems in terms of drug safety. Accordingly, to facilitate removal of the solvent through solvent evaporation, the solvent is preferably a volatile solvent.

In one embodiment, the solvent may be an alkyl halide, a fatty acid ester, an ether, an aromatic hydrocarbon, an alcohol, or a mixture thereof. More specifically, the solvent may be methylene chloride, chloroform, chloroethane, trichloroethane, carbon tetrachloride, ethyl acetate, butyl acetate, acetic acid, ethyl ether, isopropyl ether, benzene, toluene, xylene, acetonitrile, isopropanol, methanol, ethanol, or a mixture thereof.

In Examples below, a method of preparing microparticles using methylene chloride as a solvent is described. In the present invention, evaporation of the solvent is performed by slowly heating so that temperature increases from a temperature before a solvent evaporation step to a temperature in a range of a boiling point ±10° C. of the solvent. According to the preparation method of the present invention, since the boiling point of methylene chloride is approximately 39.95° C., evaporation of the solvent is performed by slowly heating at a rate of 0.2 to 2° C./min, for example, 0.3 to 1.5° C./min or 0.5 to 1° C./min, so that temperature increases from room temperature to a temperature of 30 to 50° C.

In the present invention, the aqueous medium may be an aqueous solution comprising an emulsifier. As the emulsifier, known materials used for formation of stable emulsions may be used. For example, one or more selected from the group consisting of anionic surfactants (e.g., sodium oleate, sodium stearate, sodium lauryl sulfate, and the like), nonionic surfactants (e.g., polyoxyethylene sorbitan fatty ester and the like), polyoxyethylene castor oil derivatives, polyvinylpyrrolidone, polyvinyl alcohols, carboxymethyl cellulose, lecithin, gelatin, and hyaluronic acid may be used as the emulsifier. The emulsifier in the aqueous solution may be contained at a concentration of 0.01 to 10% (w/v), for example, 0.1 to 5% (w/v).

In accordance with another aspect of the present invention, provided are sustained-release drug microparticles prepared by the method of the present invention and a pharmaceutical formulation comprising the sustained-release drug microparticles. The sustained-release drug microparticles may be formulated into various pharmaceutical formulations depending on excipients added.

In one embodiment, the sustained-release drug microparticles may be formulated into an injection for parenteral administration. In this case, by adding suitable excipients, the sustained-release drug microparticles may be formulated into an aqueous or oil-based suspension. For example, when the drug microparticles are formulated into a suspension, those skilled in the art may use a dispersion medium that allows the microparticles to exhibit good dispersibility. In addition, preservatives, tonicity agents, and the like commonly included in suspensions may be added.

In one embodiment, when the sustained-release drug microparticles are formulated into an injection, the sustained-release drug microparticles may be separated from a dispersion medium and put into a vial, and may be prepared in a suspension just before administration to a patient. In accordance with yet another aspect of the present invention, provided is a kit comprising the sustained-release drug microparticles prepared according to the method of the present invention, a dispersion medium, and a syringe. Alternatively, the syringe may be filled with the sustained-release drug microparticles and a suspension, and the sustained-release drug microparticles and the suspension may be present independently in a separate compartment in the syringe.

The microparticles prepared by the method of the present invention may have an average particle size of 10 to 500 μm.

To use the microparticles as an injection, the microparticles preferably have an average particle size of 10 to 200 μm, for example, 20 to 100 μm, without being limited thereto.

Preferably, the content ratio of the drug in the total microparticles may be 10 to 40%, for example, 15 to 35%, 20 to 30%, 20 to 27%, or 20 to 24%, and may include all sub-values within the ranges, without being limited thereto.

### Advantageous Effects

According to the preparation method of the present invention, as can be seen in Examples below, an initial release amount can be easily controlled by simply adjusting the evaporation temperature of a solvent in a conventional method of preparing microparticles. In addition, since no additional process is required, a drug loading rate can be significantly increased, and since high temperature is not required, the stability of a drug that is weak to heat can be ensured.

### DESCRIPTION OF DRAWINGS

FIG. 1 shows the results of in vitro measurement of release of donepezil from microparticles in Comparative Examples 1 to 3 and Example 1.

FIG. 2 shows in vivo PK results of a control group and a test group according to Comparative Example 2 and Example 1.

FIG. 3 shows in vivo PK results of a control group and Examples 2 to 5.

### BEST MODE

Hereinafter, the present invention will be described in detail with reference to Examples and Experimental Examples. However, Examples and Experimental Examples of the present invention are provided for illustrative purposes only and should not be construed as limiting the scope and spirit of the present invention.

### EXAMPLES

### Example 1

### Preparation Example 1: Preparation of Oil-in-Water Emulsion Containing Donepezil

Polyvinyl alcohol (PVA, 90% hydrolyzed, Mw=20,000 to 30,000) was dissolved in sterilized water to prepare a 0.5% w/v PVA aqueous solution.

Meanwhile, donepezil, methylene chloride, and polylactide (Resomer™ R203H, poly(D,L-lactide), Mw=18,000 to 24,000) were added to a beaker, and were completely dissolved by stirring to prepare a polymer/drug solution.

The polymer/drug solution was added to the PVA aqueous solution, and stirring was performed to generate an oil-in-water (O/W) emulsion.

The resulting oil-in-water (O/W) emulsion was added to the PVA aqueous solution, and re-stirring was performed once to minimize loss of drug content. In this case, the number of repetitions is not limited to one time.

### Preparation Example 2: Preparation of Microparticles Containing Donepezil

Donepezil-containing microparticles were formed from the oil-in-water emulsion of Preparation Example 1 through solvent evaporation. At this time, microparticles of Comparative Examples 1 to 3 and Example 1 were formed according to setting of temperature conditions for solvent evaporation. The formed microparticles were centrifuged at 1500 rpm for 5 minutes, lyophilized overnight, and then sieved using a 100 mesh sieve (180 μm to 80 μm, 125 μm).

TABLE 1
Preparation of microparticles of Comparative
Examples 1 to 3 or Example 1 according to setting of
temperature conditions for solvent evaporation.

| | | |
|---|---|---|
| Comparative Example 1 | Temperature | 45° C. |
| | N₂ purge | ON |
| | RPM | 120 |
| | Time | 4 hours |
| | Retention time | 2 hours |
| Comparative Example 2 | Temperature | 45° C. |
| | N₂ purge | OFF |
| | RPM | 120 |
| | Time | 4 hours |
| | Retention time | 2 hours |
| Comparative Example 3 | Temperature | 35° C. |
| | N₂ purge | OFF |
| | RPM | 120 |
| | Time | 4 hours |
| | Retention time | 2 hours |
| Example 1 | Temperature | 25° C.->45° C. |
| | N₂ purge | OFF |
| | RPM | 120 |
| | Time | 4 hours |
| | Retention time | 2 hours |

The method of controlling temperature in Preparation Examples is as follows.

1) A circulator for temperature control was attached to a stainless steel reactor in the form of a water jacket capable of circulating water. When volatilization was performed at 45° C., the temperature of the circulator was set to 45° C., and when temperature reached 45° C., an emulsified solution (microparticles in PVA solution) was added to the reactor and stirred for 4 hours.

2) In the case of 35° C., the temperature of the circulator was set to 35° C., and when temperature reached 35° C., a solution was added and stirred for 4 hours.

3) When gradually increasing temperature from 25° C. to 45° C., the temperature of the circulator was set to 25° C., and when temperature reached 25° C., a solution was added. At this time, the temperature of the circulator was set to 45° C. Time taken for temperature to increase from 25° C. to 45° C. was about 40 minutes. After 3 hours including this 40 minutes, the temperature of the circulator was set to 25° C. (taking about 1 hour). Time taken for volatilization was 4 hours in total.

### Examples 2 to 5: Preparation of Donepezil-Containing Microparticles

One-month drug release-type microparticles were prepared, according to the composition of Table 2 below, in the same manner as the method of preparing microparticles of Example 1, except that PLGA RG752H (PLA:PGA=75:25) or PLGA RG502H (PLA:PGA=50:50) is blended with PLA polymer in a ratio of 10 to 25% instead of using a PLA polymer alone.

TABLE 2
Compositions of Examples 1 to 5.

| | | | | | |
|---|---|---|---|---|---|
| DPZ300IM | Example 1 | Example 2 | Example 3 | Example 4 | Example 5 |

| | |
|---|---|
| Batch size | 30 |
| (vials) | |

| | | | | | | |
|---|---|---|---|---|---|---|
| Donepezil-free | 300 | 300 | 300 | 300 | 300 | mg/vial |
| base | | | | | | |
| Methylene | 5,000 | 5,000 | 5,000 | 5,000 | 5,000 | mg/vial |
| chloride | | | | | | |
| R203H | 950 | 855 | 760 | 855 | 760 | mg/vial |
| RG502H | — | 95 | 190 | — | — | mg/vial |
| RG752H | — | — | — | 95 | 190 | mg/vial |
| Microsphere | 1,250 | 1,250 | 1,250 | 1,250 | 1,250 | mg/vial |

### Example 2

Donepezil, methylene chloride, polylactide (Resomer™ R203H, poly(D,L-lactide), Mw=18,000 to 24,000), and PLGA RG502H (PLA:PGA=50:50) (RG502H 10% blending) were added to a beaker in amounts shown in Table 2 (Example 2), and were completely dissolved by stirring to prepare a polymer/drug solution.

Donepezil-containing microparticles were formed using the oil-in-water emulsion method of Preparation Example 1. In this case, setting of temperature conditions for solvent evaporation was the same as that of Example 1. The formed microparticles were centrifuged at 1500 rpm for 5 minutes, lyophilized overnight, and then sieved using a 100 mesh sieve (180 μm to 80 μm, 125 μm).

### Example 3

Donepezil, methylene chloride, polylactide (Resomer™ R203H, poly(D,L-lactide), Mw=18,000 to 24,000), and PLGA RG502H (PLA:PGA=50:50) (RG502H 20% blending) were added to a beaker in amounts shown in Table 2 (Example 3), and were completely dissolved by stirring to prepare a polymer/drug solution.

Donepezil-containing microparticles were formed using the oil-in-water emulsion method of Preparation Example 1. In this case, setting of temperature conditions for solvent evaporation was the same as that of Example 1. The formed microparticles were centrifuged at 1500 rpm for 5 minutes, lyophilized overnight, and then sieved using a 100 mesh sieve (180 μm to 80 μm, 125 μm).

### Example 4

Donepezil, methylene chloride, polylactide (Resomer™ R203H, poly(D,L-lactide), Mw=18,000 to 24,000), and PLGA RG752H (PLA:PGA=75:25) (RG752H 10% blending) were added to a beaker in amounts shown in Table 2 (Example 4), and were completely dissolved by stirring to prepare a polymer/drug solution.

Donepezil-containing microparticles were formed using the oil-in-water emulsion method of Preparation Example 1. In this case, setting of temperature conditions for solvent evaporation was the same as that of Example 1. The formed microparticles were centrifuged at 1500 rpm for 5 minutes, lyophilized overnight, and then sieved using a 100 mesh sieve (180 μm to 80 μm, 125 μm).

### Example 5

Donepezil, methylene chloride, polylactide (Resomer™ R203H, poly(D,L-lactide), Mw=18,000 to 24,000), and PLGA RG752H (PLA:PGA=75:25) (RG752H 20% blending) were added to a beaker in amounts shown in Table 2 (Example 5), and were completely dissolved by stirring to prepare a polymer/drug solution.

Donepezil-containing microparticles were formed using the oil-in-water emulsion method of Preparation Example 1. In this case, setting of temperature conditions for solvent evaporation was the same as that of Example 1. The formed microparticles were centrifuged at 1500 rpm for 5 minutes, lyophilized overnight, and then sieved using a 100 mesh sieve (180 μm to 80 μm, 125 μm).

### Experimental Example 1: Measurement of Drug Initial Release Rate

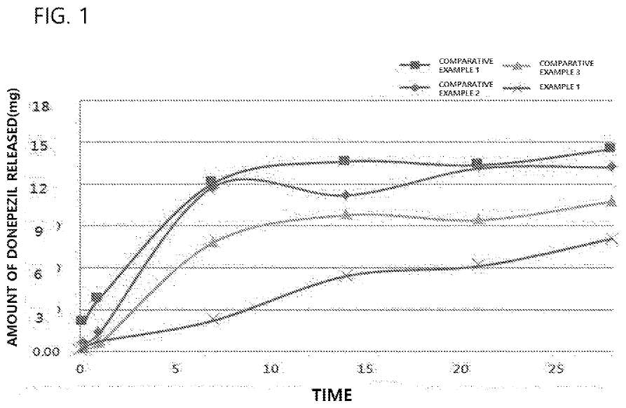

The release pattern of donepezil from the microparticles of Comparative Examples 1 to 3 and Example 1 was measured in vitro. For each group, 10 mg of microparticles (containing about 2.4 mg of donepezil) was taken. The microparticles were introduced into a discharge tube, a buffer was added thereto, and continuous shaking was performed at 100 rpm to release donepezil. The amount of donepezil released was measured using UPLC at regular time intervals, and the measured amount was converted based on 1,250 mg (containing about 300 mg of donepezil), which was the total amount of microparticles included in one vial, and the converted value is shown in FIG. 1.

As a result, as shown in FIG. 1, the microparticles of Example 1, from which a solvent was evaporated by heating over different times, exhibited the lowest initial donepezil release rate.

### Experimental Example 2: In Vivo PK Test

The release pattern of donepezil from the microparticles of Comparative Example 2 and Example 1 was measured through an in vivo PK test.

Oral donepezil hydrochloride (reference drug) was orally administered to male beagles three times at 24 hour intervals for 3 days, and the male beagles were set as a control group. The dose of donepezil per administration was 3 mg/head with a volume of 1 ml/head.

The microparticles of each of Comparative Example 2 and Example 1 were suspended in a solution containing, per 1 ml of the solution, 50 mg of D-mannitol, 5 mg of carboxymethyl cellulose sodium, 80 drops of polysorbate, and a proper amount of water for injection. The intramuscular dose of donepezil was about 90 mg/head per administration with a volume of 3 ml/head. Four experimental animals (n=4) were used for each group.

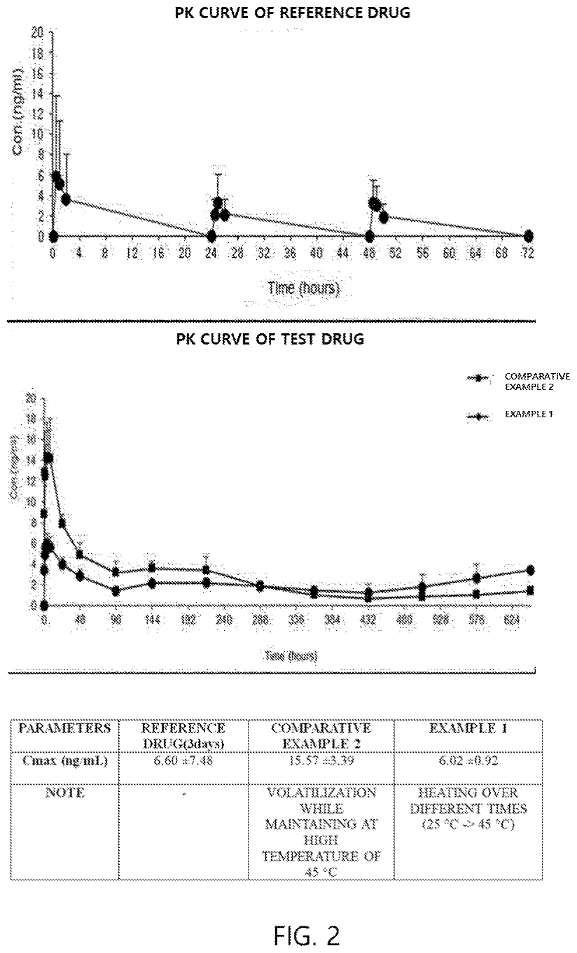

FIG. 2 shows the results of in vivo PK tests of Comparative Example 2 and Example 1, as compared with a control group. As shown in FIG. 2, the microparticles of Example 1 exhibit a Cmax similar to that of the orally administered reference drug, and the initial release rate of donepezil is also similar to that of the reference drug.

### Experimental Example 3: In Vivo PK Test

The release pattern of donepezil from the microparticles of Examples 2 to 5 was measured through an in vivo PK test.

Oral donepezil hydrochloride (reference drug) was orally administered to male beagles three times at 24 hour intervals for 3 days, and the male beagles were set as a control group. The dose of donepezil per administration was 1.24 mg/head with a volume of 1 ml/head.

The microparticles of each of Examples 2 to 5 were suspended in a solution containing, per 1 ml of the solution, 50 mg of D-mannitol, 5 mg of carboxymethyl cellulose sodium, 80 drops of polysorbate, and a proper amount of water for injection. The intramuscular dose of donepezil was about 37.2 mg/head per administration with a volume of 0.6 ml/head. Four experimental animals (n=4) were used for each group.

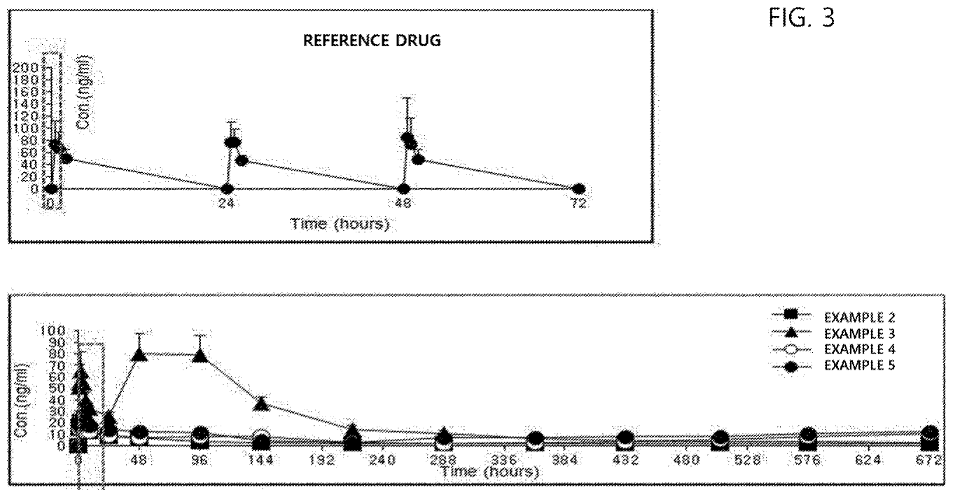

FIG. 3 shows the results of in vivo PK tests of Examples 2 to 5, as compared with a control group. The same pattern is observed for a reference drug administered orally for 3 days. Based on this result, it is expected that the same result will be obtained even if administered daily for 30 days. As shown in FIG. 3, the microparticles of Examples 2 to 5 exhibit a Cmax similar to that of the orally administered reference drug, and the initial release rate of donepezil (release within about 7 days) is also similar to that of the reference drug.

1. A method of preparing sustained-release drug microparticles, the method comprising:
mixing a mixed solution of a drug and a biodegradable polymer dissolved in a solvent with an aqueous medium to obtain an emulsion; and
evaporating the solvent from the emulsion to form microparticles containing the drug,
wherein evaporation of the solvent is performed by heating at a rate of 0.2 to 2° C./min so that temperature increases from a temperature before the solvent evaporation to a temperature in a range of a boiling point ±10° C. of the solvent.

2. The method according to claim 1, wherein the biodegradable polymer is selected from the group consisting of polylactide (PLA), polyglycolide (PGA), poly(lactide-co-glycolide) (PLGA), and mixtures thereof.

3. The method according to claim 1, wherein the biodegradable polymer has a weight average molecular weight of 4,000 to 50,000.

4. The method according to claim 1, wherein the drug is a poorly soluble drug.

5. The method according to claim 1, wherein the drug includes one or more selected from the group consisting of progesterone, haloperidol, thiothixene, olanzapine, clozapine, bromperidol, pimozide, risperidone, ziprasidone, diazepam, ethyl loflazepate, alprazolam, nemonapride, fluoxetine, sertraline, venlafaxine, donepezil, tacrine, galantamine, rivastigmine, selegiline, ropinirole, pergolide, trihexyphenidyl, bromocriptine, benztropine, colchicine, nordazepam, etizolam, bromazepam, clotiazepam, mexazolam, buspirone, goserelin, leuprolide, octreotide, cetrorelix, fluconazole, itraconazole, mizoribine, cyclosporin, tacrolimus, naloxone, naltrexone, cladribine, chlorambucil, tretinoin, carmustine, anagrelide, doxorubicin, anastrozole, idarubicin, cisplatin, dactinomycin, docetaxel, paclitaxel, raltitrexed, epirubicin, letrozole, mefloquine, primaquine, oxybutynin, tolterodine, allylestrenol, lovastatin, simvastatin, pravastatin, atorvastatin, alendronate, raloxifene, oxandrolone, estradiol, ethinylestradiol, etonogestrel, and levonorgestrel.

6. The method according to claim 1, wherein the drug is donepezil.

7. The method according to claim 1, wherein the solvent is a volatile solvent.

8. The method according to claim 1, wherein the solvent is an alkyl halide, a fatty acid ester, an ether, an aromatic hydrocarbon, an alcohol, or a mixture thereof.

9. The method according to claim 1, wherein the solvent is methylene chloride, chloroform, chloroethane, trichloroethane, carbon tetrachloride, ethyl acetate, butyl acetate, acetic acid, ethyl ether, isopropyl ether, benzene, toluene, xylene, methanol, isopropanol, acetonitrile, ethanol, or a mixture thereof.

10. The method according to claim 1, wherein the aqueous medium is an aqueous solution comprising an emulsifier.

11. The method according to claim 1, wherein the solvent is methylene chloride, and evaporation of the solvent is performed by heating at a rate of 0.2 to 2° C./min so that temperature increases from room temperature to a temperature of 30 to 50° C.

12. The method according to claim 1, wherein the microparticles have an average particle size of 10 to 500 μm.

13. Sustained-release drug microparticles obtained by the preparation method of claim 1.

14. A pharmaceutical formulation comprising the sustained-release drug microparticles of claim 13.

15. The pharmaceutical formulation according to claim 14, wherein, in comparison with an oral pharmaceutical formulation wherein an active ingredient dose identical to that of the pharmaceutical formulation comprising the sustained-release drug microparticles is administered multiple times, the pharmaceutical formulation comprising the sustained-release drug microparticles exhibits a maximum drug concentration in blood (Cmax) that is biologically equivalent to that of the oral pharmaceutical formulation.

16. A kit comprising the sustained-release drug microparticles of claim 13, a dispersion medium, and a syringe.
